(19) United States
(12) Patent Application Publication
(10) Pub. No.: US 2019/0271702 A1
(43) Pub. Date: Sep. 5, 2019

(54) METHODS OF TREATEMENT OF CANCER DISEASE BY TARGETTING TUMOR ASSOCIATED MACROPHAGE

(71) Applicants: INSERM (Institut National de la Sante et de la Recherche Medicale), Paris (FR); Universite Paul Sabatier Toulouse III, Toulouse (FR); Centre National de la Recherche Scientifique (CNRS), Paris (FR)

(72) Inventors: Mary POUPOT, Toulouse Cedex 1 (FR); Loïc YSEBAERT, Toulouse Cedex 1 (FR); Marie TOSOLINI, Toulouse Cedex 1 (FR); Jean-Jacques FOURNIE, Toulouse Cedex 1 (FR)

(21) Appl. No.: 16/319,948

(22) PCT Filed: Jul. 28, 2017
(86) PCT No.: PCT/EP2017/069154

§ 371 Date: Jan. 23, 2019

(30) Foreign Application Priority Data
Jul. 28, 2016 (EP) 16305979.3

(51) Int. Cl. G01N 33/574; A61P 35/00; A61P 35/02; C07K 16/18; C12N 15/113

(52) U.S. Cl. CPC G01N 33/57492; A61P 35/00; A61P 35/02; C07K 16/18; C12N 2310/12; G01N 2333/70596; C07K 2317/76; C12N 2310/14; C12N 2310/11; C12N 15/113

(57) ABSTRACT

The present invention relates to methods of treatment of cancer by targeting tumor associated macrophages. The inventors investigated specific marker exposed on the surface of the macrophage associated tumor in order to detect and target TAMs. They showed that sideroflexin 3, which is absent in normal macrophage, is expressed by tumors associated macrophage. The relates to a method for identifying tumor-associated macrophage (TAM) in a sample comprising the steps of i) detecting the cell surface expression of CD163, CD68, and SFXN3 markers on the cell population contained in the sample and ii) concluding that the cells expressing CD163, CD68, and SFXN3 markers are the TAMs.

### FIELD OF THE INVENTION

The present invention relates to methods for identifying tumor associated macrophage (TAMs) in a sample by detecting the cell surface expression of CD163, CD68, and SFXN3 markers. The present invention also relates to antagonist of sidoreflexin 3, for use in the treatment of cancer.

### BACKGROUND OF THE INVENTION

The importance of the tumor microenvironment in promoting cancer initiation and tumor growth has been increasingly recognized over the past decade (H. Korkaya JCI, 121, (10), 3804-3809, 2011; E. Lonardo, J. Frias-Aldeguer, Cell Cycle, 11(7), 1282-1290, 2012; J. W. Pollard, Nature Reviews Immunology, 9 (4), 259-270, 2009).

The tumor microenvironment is characterized by chronic inflammation, which, instead of inhibiting tumor growth, favors tumor formation by stimulating cell proliferation, activating Cancer stem cells (CSCs), and promoting metastasis [V. Plaks, Cell Stem Cell, 16(3), 225-238, 2015; S. M. Cabarcas, L. A. International Journal of Cancer, 129(10), 2315-2327, 2011). Leading the tumor inflammatory response are tumor associated macrophages (TAMs) [R. Noy Immunity, 41(1), 49-61, 2014]. A correlation between high numbers of TAMs and rapid disease progression and poor patient outcome has been observed for decades [L. Bingle, Journal of Pathology, 196 (3), 254-265, 2002; B.-Z. Qian Cell, 141(1), 39-51, 2010]; however, only recently was this paradoxical phenotype explained. It is now understand that this correlation is due to TAM-mediated paracrine signaling, in which macrophage derived factors activate the CSC compartment and promote stemlike features of CSCs, exacerbating tumor progression, metastasis, and even CSC chemoresistance.

Monocyte infiltration into a tumor is mediated by chemokines (e.g., CCL2, CCL5, and CXCL12), CSF-1, and components of the complement cascade [E. Bonavita, Advances in Cancer Research, 128, 141-171, 2015; E. Bonavita, Cell, 160(4), 700-714, 2015]. Once they are within the tumor, the tumor environment rapidly promotes their differentiation into tumor-conditioned macrophages. TAMs were initially believed to be biased away from an M1 phenotype, expressing M2 protumor markers [Biswas S K. Nature Immunology, 11(10), 889-896, 2010].

To more specifically identify M2-like TAMs and subsets, the hemoglobin-scavenger receptor CD163 [Heusinkveld M. The Journal of Immunology, 187(3), 1157-1165, 2011; Martinez F O., Annual Review of Immunology, 27, 451-483, 2009], the macrophage scavenger receptor 1 CD204 [Biswas S K. Nature Immunology, 11(10), 889-896, 2010, Laoui, D. International Journal of Developmental Biology, 55(7-9), 861-867, 2011], the mannose receptor CD206 [Mantovani, A. Trends in Immunology, 23(11), 549-555, 2002], the macrophage receptor CD68 (Tang X, Cancer Letter 2013, Takeuchi H, Oncol Lett 2016; Hu H, tumour biol 2016; Kim K J, PlosOne 2015) and more recently the T-cell immunoglobulin andmucin-domain containing protein-3 (Tim-3) [Yan W., Gut, 64(10), 1593-1604, 2015] have been used with great success. Ultimately, however, there remains considerable controversy regarding how to properly classify and identify TAMs. While classifications based on TAM functions, such as the promotion of angiogenesis or immunosuppression, are now being used to better categorize TAMs, it is important to note that macrophages are dynamic, plastic cells capable of performing many functions simultaneously.

Thus, this approach may be self-limiting and underscore the multifunctional capabilities of TAMs. Since the scientific community has yet to come to a consensus regarding what markers to use and how to refer to macrophages, the binary M1/M2 classification remains commonly used [Murray, P J. Immunity, 41(1), 14-20, 2014].

Therefore, there is a need for new biological markers of TAMs. In particular, biomarkers that would allow reliable detection and also targeting TAM present in the tumour are highly desirable.

The purpose of the present invention is therefore to address this need by providing: i) a new reliable method for identifying tumor associated macrophage at an early stage of the disease onset and ii) a new therapeutic target for treating cancer by depleting TAM.

### SUMMARY OF THE INVENTION

A first object of the invention relates to a method for identifying tumor-associated macrophage (TAMs) in a sample comprising the steps of i) detecting the cell surface expression of CD163, CD68, and SFXN3 markers on the cell population contained in the sample and ii) concluding that the cells expressing CD163, CD68, and SFXN3 markers are the TAMs.

A second object of the invention relates SFXN3 antagonist for use in the prevention or treatment of a patient affected with a cancer.

### DETAILED DESCRIPTION OF THE INVENTION

The present invention is based on the identification of the presence of sideroflexin 3 in tumor-associated macrophages (TAMs also called, Nurse like Cells (NLCs) in leukemia), which has never been mentioned in the prior art.

More particularly, inventors present evidence that sideroflexin 3, which is absent in normal macrophage, is expressed by tumors associated macrophage. This is the first specific marker that is exposed on the surface of the macrophage associated tumors, and as a consequence offers unique opportunities for detecting and targeting TAM (for therapy of cancer). The inventors further present evidence that using antibody directed sideroflexin 3, they depleted in PBMC sample obtained from LCC patient, TAMs and strongly reduced leukemic B cells number.

Here the inventors investigated specific marker exposed on the surface of the macrophage associated tumor in order to detect and target TAMs. By immunizing mice with TAMs obtained from LCC patients they recover, between 200 fractions, four antibodies fractions able to recognize specifically TAMs. They found surprisingly that 1) antibodies in the fractions were able to recognize the sideroflexin 3 protein specifically expressed at the surface of macrophage associated tumors 2) antibodies were specific to the TAMs and did not recognized other PBMCs from patients or healthy donors (like lymphocytes B and T), or tumour cells 3) use of sideroflexin 3 antibodies in PBMC sample obtained from LCC patient, depleted TAMs and reduced tumor cells number and 4) sideroflexin 3 antibodies were also able to detect TAMs present in breast tumor sample. These results show that targeting sideroflexin 3 expressed on TAMS therefore allows to restore beneficial anti-tumor immunity in cancer.

Method for identifying tumor associated macrophage (TAM) An object of the present invention relates to a method for identifying tumor associated macrophage (TAM) in a sample comprising the steps of i) detecting the cell surface expression of CD163, CD68, and SFXN3 markers on the cell population contained in the sample and ii) concluding that the cells expressing CD163, CD68, and SFXN3 are the tumor associated macrophages.

The term“tumor associated macrophage” (also called “TAM”) has its general meaning in the art and is intended to describe a type of cell belonging to the macrophage lineage. They are found in close proximity or within tumor masses. [Shih, J-Y., Journal of Cancer Molecules 2(3): 101-106 2006] TAMs are derived from circulating monocytes or resident tissue macrophages, which form the major leukocytic infiltrate found within the stroma of many tumor types. There is growing evidence for their involvement in pro-tumor (e.g. promotion of growth and metastasis through tumor angiogenesis) processes. [Birbrair, A. American Journal of Physiology. Cell Physiology 307 (1): C25-C38, 2014; Thoreau, M; Oncotarget 6(29) 27832-27832 2015] TAMs interact with a wide range of growth factors, cytokines and chemokines in the tumor microenvironment which is thought to educate the TAMs and determine their specific phenotype and hence functional role as the microenvironment varies between different types of tumors. TAMs have therefore been shown to differ in their roles depending on the type of tumor with which they are associated [Lewis, C E.; Cancer Research 66 (2): 605-612. (2006)]. In many tumor types TAM infiltration level has been shown to be of significant prognostic value. TAMs have been linked to poor prognosis in breast cancer, ovarian cancer, types of glioma and lymphoma; better prognosis in colon and stomach cancers and both poor and better prognoses in lung and prostate cancers. [Allavena, P. Critical Reviews in Oncology/Hematology 66: 1. (2008)]. In leukemia TAMs are also called Nurse like Cells (NLCs).

The demonstration of regulatory/suppressive function of TAM cells may be determined by any suitable method known in the art (see Qian B Z and Pollard J W, Cell, 2010, vol 141, 1:39-41). In particular, examples of such tests are set out in the example section. Specifically, the tests embodied in example are regarded as standards in vitro tests for the assessment of TAM function.

In some embodiments, tumor associated macrophage according to the present invention are mammalian tumor associated macrophage, most particularly human Tumor associated macrophage.

The term “sample” refers to fluid sample and “tissue sample”

The term “fluid sample” refers to any sample which is susceptible to contain a population of Tumor associated macrophage in suspension. Non-limiting examples include biological fluids such as blood (e.g., peripheral blood or umbilical cord blood), urine, lymph, cerebral spinal fluid, or ductal fluid, or such fluids diluted in a physiological solution (e.g., saline, phosphate-buffered saline (PBS), or tissue culture medium), or cells obtained from biological fluids (e.g., by centrifugation) and suspended in a physiological solution. Other examples of a “fluid sample containing cells” include cell suspensions (in physiological solutions) obtained from bone marrow aspirates, needle biopsy aspirates or biopsy specimens from, for example, lymph node or spleen.

In some embodiments, the fluid sample is a blood sample. The term “blood sample” means a whole blood sample obtained from a subject (e.g. an individual for which it is interesting to determine whether a population of Tumor associated macrophage can be identified).

In some embodiments, the fluid sample is a PBMC sample. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, the PBMC sample may have been subjected to a selection step to contain non-adherent PBMC (which contain T cells, B cells, natural killer (NK) cells, NK T cells and DC precursors). A PBMC sample according to the invention therefore contains lymphocytes (B cells, T cells, NK cells, NKT cells). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known to the expert in the art.

In some embodiments, the fluid sample is a sample of tumor associated macrophage in suspension. Typically, the sample of tumor associated macrophage is prepared by FACS sorting methods preformed on a PBMC sample. For example, tumor associated macrophage are isolated by using antibodies for TAM associated cell surface markers, CD163, and CD68. When the method is performed from a sample of tumor associated macrophage, only the detection of the cell surface expression of SFXN3 can be carried out. Hence, in one aspect, the invention relates to a method for identifying tumor associated macrophage comprising the steps consisting of i) of selecting the population of CD163+/CD68 cells (“tumor associated macrophage”) from a PBMC sample and ii) identifying the population of tumor associated macrophage by detecting the cell surface expression of SFXN3.

As used herein, the term “CD163” (Cluster of Differentiation 163) also known as “M130”; “MM130”; “SCARII” has its general meaning in the art and refers to a protein that in humans is encoded by the CD163 gene. [Gene ID: 9332] CD163 is exclusively expressed in monocytes and macrophages. It functions as an acute phase-regulated receptor involved in the clearance and endocytosis of hemoglobin/haptoglobin complexes by macrophages, and may thereby protect tissues from free hemoglobin-mediated oxidative damage. This protein may also function as an innate immune sensor for bacteria and inducer of local inflammation. The molecular size is 130 kDa. The receptor belongs to the scavenger receptor cysteine rich family type B and consists of an 1048 amino acid residues extracellular domain, a single transmembrane segment and a cytoplasmic tail with several splice variants.

As used herein, the term “CD68” refers (Cluster of Differentiation 68) also known as “GP110”; “LAMP4” and “SCARD1” has its general meaning in the art and refers to a protein that in humans is encoded by the CD68 gene. [Gene ID: 968]. This gene encodes a 110-kD transmembrane glycoprotein that is highly expressed by human monocytes and tissue macrophages. It is a member of the lysosomal/endosomal-associated membrane glycoprotein (LAMP) family. The protein primarily localizes to lysosomes and endosomes with a smaller fraction circulating to the cell surface. It is a type I integral membrane protein with a heavily glycosylated extracellular domain and binds to tissue- and organ-specific lectins or selectins. The protein is also a member of the scavenger receptor family. Scavenger receptors typically function to clear cellular debris, promote phagocytosis, and mediate the recruitment and activation of macrophages.

As used herein, the term “sideroflexin 3” or “SFXN3”, also known as “SFX3;”, or “BA108L7.2” is a one member of Sideroflexin proteins and refers to a protein that in humans is encoded by the SFXN3 gene. [Gene ID: 81855]. Sideroflexin is commonly referred to as proteins involved in iron transport in mitochondria. In addition, the human Sideroflexin is also reported to play an important role in the differentiation of pancreatic β cells (Yoshikumi Y, J Cell Biochem., 95, 1157-1168 (2005)). One example of wild-type sideroflexin 3 human amino acid sequence is provided in SEQ ID NO:1 (NCBI Reference Sequence: NP_112233). One example of nucleotide sequence encoding wild-type sideroflexin 3 amino acid sequence of SEQ ID NO:1 is provided in SEQ ID NO:2 (NCBI Reference Sequence: NM_030971).

Standard methods for detecting the expression of a specific surface marker such as Sideroflexin 3 at cell surface (e.g. TAM surface) are well known in the art. Typically, the step consisting of detecting the surface expression of a surface marker (e.g. Sideroflexin 3) may consist in using at least one differential binding partner directed against the surface marker, wherein said cells are bound by said binding partners to said surface marker.

As used herein, the term “binding partner directed against the surface marker” refers to any molecule (natural or not) that is able to bind the surface marker with high affinity. The binding partners may be antibodies that may be polyclonal or monoclonal, preferably monoclonal antibodies. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

For example, the binding partner of Sideroflexin 3 of the invention is the anti human Sideroflexin 3 antibody available from Abnova (Purified Mouse Anti-Human Sideroflexin 3 Clone 4A3) or is selected from the group consisting of the antibodies available from Abcam (Clone ab77431 or clone ab181163).

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or a radioactive agent to the antibody or aptamer, as well as indirect labelling of the probe or antibody by reactivity with a detectable substance. An antibody or aptamer of the invention may be labelled with a radioactive molecule by any method known in the art. More particularly, the antibodies are already conjugated to a fluorophore (e.g. FITC-conjugated and/or PE-conjugated).

The aforementioned assays may involve the binding of the binding partners (ie. antibodies or aptamers) to a solid support. The solid surface could a microtitration plate coated with the binding partner for the surface marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.). According to the invention, methods of flow cytometry are preferred methods for detecting the surface expression of the surface markers (i.e. CD163, CD68 and Sideroflexin 3). Said methods are well known in the art. For example, fluorescence activated cell sorting (FACS) may be therefore used. Cell sorting protocols using fluorescent labeled antibodies directed against the surface marker (or immunobeads coated with antibody) in combination with antibodies directed against CD163, CD68, and Sideroflexin 3 coupled with distinct fluorochromes (or immunobeads coated with anti CD163 antibodies, anti CD68 antibodies and Sideroflexin 3 antibodies) can allow direct sorting, using cell sorters with the adequate optic configuration.

A further object of the present invention relates to a method for identifying tumor associated macrophage (TAM) in a tissue sample comprising the steps of i) detecting the cell expression of CD163, CD68 and Sideroflexin 3 markers and ii) concluding that the cells expressing CD163, CD68 and Sideroflexin 3 are the tumor associated macrophage.

As used herein, the term “tissue sample” refers to a sample that is typically made up of a collection of biological cells and includes, but is not limited to, for example, biopsy samples, autopsy samples, surgical samples, cell smears, cell concentrates and cultured cells fixed on a support. Typically, the tissue sample generally includes any material for which microscopic examination of samples of the material prepared on microscope slides is desirable. The tissue sample may be collected for diagnostic, research, teaching or other purposes. The sample may be of any biological tissue. Examples of tissue samples include, but are not limited to, tissue sections of brain, adrenal glands, colon, small intestines, stomach, heart, liver, skin, kidney, lung, pancreas, testis, ovary, prostate, uterus, thyroid and spleen. The “tissue sample” as used herein may be sections of tissues that are either fresh, or frozen, or fixed and embedded. For example, tissue samples for histological examination are embedded in a support medium and moulded into standardized blocks. Paraffin wax is a known and commonly-used as a support medium, however it will be appreciated that other support media, including but not limited to, TissueTek O.C.T., manufactured by Sakura Finetek, ester, microcrystalline cellulose, bees wax, resins or polymers, such as methacrylates, may also be used as support media. Suitable resins and polymers, including Araldite 502 Kit, Eponate 12™, Kit, and Glycol Methacrylate (GMA) Kit, are available from Ted Pella, Inc., Redding, Calif.

In some embodiments, the tissue sample is a tumor sample. A “tumor sample” is a sample containing tumor material e.g. tissue material from a neoplastic lesion taken by aspiration or puncture, excision or by any other surgical method leading to biopsy or resected cellular material, including preserved material such as fresh frozen material, formalin fixed material, paraffin embedded material and the like. Such a biological sample may comprise cells obtained from a patient. The cells may be found in a cell “smear” collected, for example, by a nipple aspiration, ductal lavage, fine needle biopsy or from provoked or spontaneous nipple discharge.

In some embodiments, the tissue sample is a tissue sample selected from the group consisting of tissue sections of brain, head and neck, adrenal glands, colon, small intestines, stomach, heart, liver, skin, kidney, lung, pancreas, testis, ovary, prostate, uterus, thyroid, spleen, bladder, breast, bone marrow and lymph nodes.

The detection of the cell expression of CD163, CD68, and SFXN3 markers in the tissue sample is performed by immunochemistry or immunofluorescence.

Typically, the detection of the markers in the tissue sample is performed with a IHC method. An immunohistochemistry (IHC) method is indeed suitable for detecting the present tumor associated macrophage in a tissue sample. IHC specifically provides a method of detecting targets in a tissue specimen in situ. The overall cellular integrity of the sample is maintained in IHC, thus allowing detection of both the presence and location of the targets of interest (i.e. the Tumor associated macrophage). Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). In particular embodiment, a tissue section (e.g. a sample comprising tumor associated macrophage) may be mounted on a slide or other support after incubation with antibodies directed against the CD163, CD68 and Sideroflexin 3 markers. Then, microscopic inspections in the sample mounted on a suitable solid support may be performed. For the production of photomicrographs, sections comprising samples may be mounted on a glass slide or other planar support, to highlight by selective staining the presence of the proteins of interest. Therefore IHC samples may include, for instance: (a) preparations of the tissue sample (b) fixed and embedded said cells and (c) detecting the proteins of interest in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

In some embodiments, the method of the invention further comprises a step consisting of determining the level of tumor associated macrophage present in the sample.

### Therapeutic Methods and Uses

The present invention provides methods and compositions (such as pharmaceutical compositions) for preventing or treating a cancer. The present invention also provides methods and compositions for inhibiting or preventing cancer.

In the context of the invention, the term “treatment or prevention” means reversing, alleviating, inhibiting the progress of, or preventing the disorder or condition to which such term applies, or one or more symptoms of such disorder or condition. In particular, the treatment of the disorder may consist in reducing the number of malignant cells. Most preferably, such treatment leads to the complete depletion of the malignant cells.

Preferably, the individual to be treated is a human or non-human mammal (such as a rodent (mouse, rat), a feline, a canine, or a primate) affected or likely to be affected with cancer. Preferably, the individual is a human.

According to a first aspect, the present invention relates to a sideroflexin 3 antagonist for use in the prevention or the treatment of a patient affected with a cancer.

The terms “cancer” “malignancy” and “tumors” refer to or describe the pathological condition in mammals that is typically characterized by unregulated cell growth. More precisely, in the use of the invention, diseases, namely cancer are associated with tumor-associated macrophages TAMs, which have been shown to be symbiotically related to tumor cells. Furthermore, inventors show that antagonist of the invention, like antibodies directed to sidoreflexin 3, kill Tumor-associated macrophages (TAMs) and decrease tumor cell growth: Tumor cells recruit TAMs which provide these with survival and angiogenic factors in the tumor microenvironment (see review Dirkx A. E. M. Journal of Leukocyte Biology vol. 80 no. 6 1183-1196 December 2006).

In particular, the cancer may be associated with a solid tumor or unregulated growth of undifferentiated bone marrow cells (i.e. leukemia, lymphoma).

A variety of cancers and other proliferative diseases including, but not limited to the following can be treated using the methods and compositions of the invention:
carcinoma, including that of the bladder, breast, uterine/cervical, colon, kidney, liver, lung, ovary, oesophage, pancreas, prostate, stomach, cervix, thyroid, colorectal, head and neck and skin, including squamous cell carcinoma,
tumors of mesenchymal origin, including fibrosarcoma and rhabdomyoscarcoma;
other tumors, including melanoma, seminoma, teratocarcinoma, neuroblastoma and glioma;
tumors of the central and peripheral nervous system, including astrocytoma, neuroblastoma, glioma, and schwannomas;
tumors of mesenchymal origin, including fibrosarcoma, rhabdomyoscaroma, and osteosarcoma; and
other tumors, including melanoma, xeroderma pigmentosum, keratoacarcinoma, seminoma, thyroid follicular cancer and teratocarcinoma.
lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma, Adult T-cell leukemia/lymphoma.
leukemias such as, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia.

In one embodiment said cancer is a leukemia, which is selected from the group consisting of all acute and chronic leukemia: chronic lymphocytic leukemia (CLL), acute myelocytic leukemias (AML), acute lymphocytic leukemia (ALL), Adult T-cell leukemia/lymphoma (ATLL), Chronic myelomonocytic leukaemia (LMMC), Acute promyelocytic leukemia (APL).

In preferred embodiment said leukemia is chronic lymphocytic leukemia (CLL),

In one embodiment said cancer is a lymphoma which is selected from the group consisting of all non-Hodgkinien or Hodgkinien lymphomas, Adult T-cell leukemia/lymphoma (ATLL)

In one embodiment said cancer is a solid tumor selected from the group consisting of brain, head and neck, adrenal glands, colon, small intestines, stomach, heart, liver, skin, kidney, lung, pancreas, testis, ovary, prostate, uterus, thyroid, bladder, breast, endometrial tumors, multiple myeloma and sarcomas.

Where hereinbefore and subsequently a tumor, a tumor disease, a carcinoma or a cancer are mentioned, metastasis in the original organ or tissue and/or in any other location are implicitly meant alternatively or in addition, whatever the location of the tumor and/or metastasis is.

The term “patient” or “subject in need thereof” refers to any mammal (preferably human) afflicted with or susceptible to be afflicted with a pathology involving adenosine receptor.

A “sideroflexin 3 antagonist” refers to a molecule (natural or synthetic) or cells capable of neutralizing, blocking, inhibiting, abrogating, reducing or interfering with the activities of sideroflexin 3. Sideroflexin 3 antagonists include antibodies and antigen-binding fragments thereof, Chimeric Antigen Receptor T cell (CAR-T), proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include, antagonist variants of the protein, siRNA molecules directed to a protein, antisense molecules directed to a protein, aptamers, and ribozymes against a protein. For instance, the sideroflexin 3 antagonists may be a molecule that binds to sideroflexin 3 and neutralizes, blocks, inhibits, abrogates, reduces or interferes with the biological activity of sideroflexin 3 when expressed on TAM surface (such as inducing tumor cell growth, and promoting metastasis). More particularly, the sideroflexin 3 antagonist according to the invention is an anti-sideroflexin 3 antibody.

By “biological activity” of sideroflexin 3 is meant, when expressed on TAMs surface, inducing tumor cell growth and/or viability, immunosuppression and promoting tumor metastasis.

Tests for determining the capacity of a compound to be sideroflexin 3 antagonist are well known to the person skilled in the art. In a preferred embodiment, the antagonist specifically binds to sideroflexin 3 in a sufficient manner to inhibit the biological activity of sideroflexin 3 when expressed on TAM surface. Binding to sideroflexin 3 and inhibition of the biological activity of sideroflexin 3 may be determined by any competing assays well known in the art. For example, the assay may consist in determining the ability of the agent to be tested as sideroflexin 3 antagonist to bind to sideroflexin 3. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an antagonist that “specifically binds to sideroflexin 3” is intended to refer to an inhibitor that binds to human sideroflexin 3 polypeptide with a KD of 1 μM or less, 100 nM or less, 10 nM or less, or 3 nM or less. Then a competitive assay may be settled to determine the ability of the agent to inhibit biological activity of sideroflexin 3. The functional assays may be envisaged such evaluating the ability to inhibit a) tumor cell growth and/or viability (see example with blocking sideroflexin 3 antibody and FIGS. 2 and 3) and/or b) tumor metastasis (test based on the effect of the antagonist on the inhibition of angiogenesis and/or tumor cells migration (Matrigel assay) may be used.

The skilled in the art can easily determine whether a sideroflexin 3 antagonist neutralizes, blocks, inhibits, abrogates, reduces or interferes with a biological activity of sideroflexin 3. To check whether the sideroflexin 3 antagonist bind to sideroflexin 3 and/or inhibit tumor cell growth in the same way than the initially characterized blocking sideroflexin 3 antibody and/or binding assay and/or a cell proliferation and/or viability assay may be performed with each antagonist. For instance cell proliferation assay can be measured by CFSE-proliferation assay. For instance cell viability assay can be measured by propiodium iodure measure by flow cytometry. For instance angiogenesis and/or tumor cells migration assay can be measured by Matrigel assay.

Accordingly, the sideroflexin 3 antagonist may be a molecule that binds to sideroflexin 3 selected from the group consisting of antibodies, aptamers, and polypeptides.

The skilled in the art can easily determine whether a sideroflexin 3 antagonist neutralizes, blocks, inhibits, abrogates, reduces or interferes with a biological activity of sideroflexin 3: (i) binding to sideroflexin 3 and/or (ii) inhibiting tumor cell growth and/or viability and/or (iii) blocking tumor metastasis.

Antibody

In another embodiment, the sideroflexin 3 antagonist is an antibody (the term including antibody fragment or portion) that can bind to sideroflexin 3 at the TAMs surface and block TAMs activity (inducing tumor cell growth and/or viability, immunosuppression and promoting tumor metastasis.

In preferred embodiment, the sideroflexin 3 antagonist may consist in an antibody directed against the sideroflexin 3, in such a way that said antibody impairs TAMs activity (“neutralizing antibody”).

Then, for this invention, neutralizing antibody of sideroflexin 3 are selected as above described for their capacity to (i) bind to sideroflexin 3 and/or (ii) inhibiting tumor cell growth and/or viability and/or (iii) blocking tumor metastasis.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of sideroflexin 3. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant sideroflexin 3 may be provided by expression with recombinant cell lines. Recombinant form of sideroflexin 3 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Example of neutralizing anti-sideroflexin 3 antibodies is disclosed in the example section. The skilled artisan can use routine technologies to use the antigen-binding sequences of these antibodies (e.g., the CDRs) and generate humanized antibodies for treatment of cancer as disclosed herein.

Chimeric Antigen Receptor T Cell (CAR-T)

In some embodiments, the antigen biding fragment of an anti-sideroflexin 3 antibody is a part of a Chimeric Antigen Receptor T cell (CAR-T).

In a particular embodiment, CAR-T cells comprises an antigen-binding domain comprising, consisting of, or consisting essentially of, a single chain variable fragment (scFv) of the invention. In some embodiments, the antigen binding domain comprises a linker peptide. The linker peptide may be positioned between the light chain variable region and the heavy chain variable region.

As used herein, the term “Chimeric Antigen Receptor T Cells” also called CAR-T Cells refers to lymphocytes which express Chimeric Antigen Receptor (CAR). The term “Chimeric Antigen Receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independently of antigen processing, thus bypassing a major mechanism of tumor escape. Moreover, when expressed in T-cells, CARs advantageously do not dimerize with endogenous T cell receptor (TCR) alpha and beta chains. Strategies to design and produce such CARs are well known in the art, references can be found for example in Bonini and Mondino, Eur. J. Immunol. 2015 (19), Srivastava and Riddell, Trends Immunol. 2015 (20), Jensen and Riddell, Curr. Opin. Immunol. 2015 (21), Gill and June, Immunol. Rev. 2015 (22).

Aptamer

In another embodiment, the sideroflexin 3 antagonist is an aptamer directed against sideroflexin 3. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of sideroflexin 3 are selected as above described for their capacity to (i) bind to sideroflexin 3 and/or (ii) inhibit tumor cell growth and/or viability and/or (iii) blocking tumor metastasis.

Inhibitor of Sideroflexin 3 Gene Expression

In still another embodiment, the sideroflexin 3 antagonist is an inhibitor of sideroflexin 3 gene expression. An “inhibitor of expression” refers to a natural or synthetic compound that has a biological effect to inhibit the expression of a gene. Therefore, an “inhibitor of sideroflexin 3 gene expression” denotes a natural or synthetic compound that has a biological effect to inhibit the expression of sideroflexin 3 gene.

In a preferred embodiment of the invention, said inhibitor of sideroflexin 3 gene expression is a siRNA, an antisense oligonucleotide, a nuclease or a ribozyme.

Inhibitors of sideroflexin 3 gene expression for use in the present invention may be based on antisense oligonucleotide constructs. Anti-sense oligonucleotides, including anti-sense RNA molecules and anti-sense DNA molecules, would act to directly block the translation of sideroflexin 3 mRNA by binding thereto and thus preventing protein translation or increasing mRNA degradation, thus decreasing the level of sideroflexin 3, and thus activity, in a cell. For example, antisense oligonucleotides of at least about 15 bases and complementary to unique regions of the mRNA transcript sequence encoding sideroflexin 3 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of sideroflexin 3 gene expression for use in the present invention. sideroflexin 3 gene expression can be reduced by using small double stranded RNA (dsRNA), or a vector or construct causing the production of a small double stranded RNA, such that sideroflexin 3 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Examples of said siRNAs against sideroflexin 3 include, but are not limited to those described in Yoshikumi Y (2005) J Cell Biochem. August 15; 95(6):1157-68.

Ribozymes can also function as inhibitors of sideroflexin 3 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of sideroflexin 3 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of sideroflexin 3 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing sideroflexin 3. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

### Method of Preventing or Treating Cancer

The present invention further contemplates a method of preventing or treating cancer in a subject comprising administering to the subject a therapeutically effective amount of a sideroflexin 3 antagonist.

In one aspect, the present invention provides a method of inhibiting tumor growth and/or viability in a subject comprising administering a therapeutically effective amount of a sideroflexin 3 antagonist.

By a “therapeutically effective amount” of a sideroflexin 3 antagonist as above described is meant a sufficient amount of the antagonist to prevent or treat a pancreatic ductal adenocarcinoma. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The invention also relates to a method for treating cancer in a subject having a high level of TAMs expressing sideroflexin 3 in blood with a sideroflexin 3 antagonist.

The invention also relates to sideroflexin 3 antagonist for use in the treatment of a cancer in a subject having a high level of TAMs expressing sideroflexin 3 in blood.

The above method and use comprise the step of measuring the level of TAMs expressing sideroflexin 3 protein in a blood sample obtained from said subject wherein and compared to a reference control value.

A high level of of TAMs expressing sideroflexin 3 is predictive of a high risk of having or developing a cancer and means that sideroflexin 3 antagonist must be used.

Typically, a body fluid sample is obtained from the subject and the level of sideroflexin 3 is measured in this sample. Indeed, statistical analyses revealed that decreasing of TAMs expressing sideroflexin 3 levels would be particularly beneficial in those patients displaying high levels of TAMs expressing sideroflexin 3.

### Pharmaceutical Compositions of the Invention

The sideroflexin 3 antagonist as described above may be combined with pharmaceutically acceptable excipients, and optionally sustained-release matrices, such as biodegradable polymers, to form therapeutic compositions.

Accordingly, the present invention relates to a pharmaceutical composition comprising a sideroflexin 3 antagonist according to the invention and a pharmaceutically acceptable carrier.

The present invention also relates to a pharmaceutical composition for use in the prevention or treatment of cancer comprising a sideroflexin 3 antagonist according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In therapeutic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of sideroflexin 3 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

Rectal administration is effected using suppositories prepared with binders which melt at the rectal temperature, for example cacao butter or polyethylene glycols.

Parenteral, intranasal or intraocular administration is effected using aqueous suspensions, isotonic saline solutions or sterile and injectable solutions which contain pharmacologically compatible dispersants and/or wetting agents, for example propylene glycol, butylene glycol, or polyethylene glycol.

Thus a cosolvent, for example an alcohol such as ethanol or a glycol such as polyethylene glycol or propylene glycol, and a hydrophilic surfactant such as Tween® 80, can be used to prepare an aqueous solution injectable by intravenous route. The active principle can be solubilized by a triglyceride or a glycerol ester to prepare an oily solution injectable by intramuscular route.

Transdermal administration is effected using multilaminated patches or reservoirs into which the active principle is in the form of an alcoholic solution.

Administration by inhalation is effected using an aerosol containing for example sorbitan trioleate or oleic acid together with trichlorofluoromethane, dichlorotetrafluoroethane or any other biologically compatible propellant gas.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

The active principle can also be presented in the form of a complex with a cyclodextrin, for example .alpha.-, .beta.- or .gamma.-cyclodextrin, 2-hydroxypropyl-.beta.-cyclodextrin or methyl-.beta.-cyclodextrin.

### FIGURES

FIG. 1. High staining of NLC by the four anti-NLC antibodies. A. Staining with the four anti-NLC antibodies of B-CLL and NLC gated from a patient with CLL and of monocytes, T cells and B cells from blood sample of an healthy donor (full line: isotype control; dashed line: antibodies). B. Positive staining with the 6-25 antibody on gated CD163 NLC and negative staining on other cells. C. Mean of the fluorescence intensity of the four antibodies staining on NLC gated compared to that of the isotype control.

FIG. 2. NLC depletion and leukemic cells death when cultured with the 4 antibodies. Visualisation by microscopy of the depletion of NLC (surrounded) in a culture of PBMC from a patient with CLL after 6 days in the presence of each anti-NLC antibodies compared to the isotype condition. The absolute number of NLC in these cultures was near to zero in the conditions with each antibody.

FIG. 3: Western blot of the recombinant sideroflexin 3 revealed by two commercial anti-SFXN3 antibodies and the 6-25 antibody or the isotype.

FIG. 4: Microscopy and B CLL viability analysis, from a 10 days culture of PBMC from CLL patient with or without 6-25 antibody or isotypic control. FACS: Percentage of dead B CCL framed.

FIG. 5: NLC numbers after 10 days of culture of CLL patient's PBMC with or without 6-25 or isotypic control.

### EXAMPLE 1: SIDEROFLEXIN TO TARGET TUMOR ASSOCIATED MACROPHAGES

Material & Methods

PBMC from patients were isolated using Ficoll and cultured 10 days at 10 10⁶ cells/ml in RPMI 10% FBS. Then supernatant were removed and adherents cells (NLC) were washed twice with PBS. NLC were detached using cell scraper, counted and dried pellets were freezed. Dry pellets of non adherent cells were also freezed. NLC from several patients were pooled and membrane proteins were extracted using the “Proteo-extract native membrane protein extraction kit” (Calbiochem).

Pierce™ High Capacity Streptavidin Agarose beads (thermo Fisher Scientific) were incubated with goat anti mouse IgG-biotin (Sigma Aldrich, B7401) 20 minutes at room temperature, washed three times and then incubated 2 hours at 4° C. with hybridoma's supernatant culture medium or with a control medium. After three PBS washes, coated beads were incubated over night at 4° C. with 500 μg of membrane proteins from NLC or non-adherent cells. Beads were washed and eluted with Laemmli solution. These solutions were loaded in an acrylamide gel and colored with instant blue (euromedex) after migration. Gel strips were cut and peptides were extracted. Briefly, strips were washed with acetonitrile and ammonium bicarbonate, reduced and alkylated with DTT and iodoacetamin, digested with trypsine and then peptides were extracted with acetonitrile and formic acid. Peptides were then identified by mass spectometry. Identified peptides in each condition (without hybridoma's supernatant, without proteins, with proteins from non-adherent cells and from NLC) were compared.

Results

To determine the target of the four antibodies which specifically recognize NLC, we tested by immunoprecitation the four hybridomas culture supematants. We thus identified by mass spectrometry the peptides associated to each hybridomas.

We Found these Peptides:
6-16: sideroflexin 3 and GRP78
6-25: sideroflexin 3
6-66-49: GRP78
6-66-74: endoplasmine and GRP78

### EXAMPLE 2: TARGETING OF TUMOR ASSOCIATED MACROPHAGES

Material & Methods

Production of Antibodies Against NLC

NLC were generated from culture of PBMC isolated from blood samples of patients with chronic lymphoid leukemia (CLL) at 10 10⁶ cells/ml in RPMI 10% FBS. After 14 days of culture at 37° C. and in a 5% C02 atmosphere, B leukemic cells were removed and adherent cells (NLC) were collected. Between 3 and 15 millions of NLC from different donors were injected to mouse in intraperitoneal four times every 15 days. Splenocytes were then isolated from the spleen and fusion with the c63s2 murine myeloma cell line produced 200 hybridomas. The 200 conditioned medium of these hybridomas cultures were incubated with NLC or leukemic cells for 30 minutes at 4° C., then with a fluorescent secondary anti-mouse antibody. These cells were then analyzed by flow cytometry.

Flow Cytometry Analysis

50 μl of each hybridoma culture supernatant were incubated with 0.2 millions of cells (NLC, B leukemic cells or PBMC from CLL′ patients or healthy donors) for 30 minutes at 4° C. After washing with PBS, cells were incubated with goat anti-mouse antibody coupled to a fluorochrome for 30 minutes at 4° C. After washing, cells were incubated with antihuman-CD163, antihuman-CD3, antihuman-CD19 and antihuman-CD14 coupled to different fluorochromes for 15 minutes at 4° C. After washing, cells were analyzed by flow cytometry.

Culture with Antibodies

PBMC isolated from a patient with CLL were cultured for 6 days with or without 5 μl/ml of purified antibodies from hybridomas culture supernatants or with 5 μl/ml of isotype control. Numbers of NLC were then estimated by a counting under microspcope and viable leukemic cells were evaluated thanks to a cell counter using propidium iodure for the viability.

Immunohistochemistry

IHC was performed on 4-μm-thick routinely processed formalin-fixed paraffin-embedded sections of tumor specimens with neighboring normal tissue. A heat-induced epitope retrieval technique in a citrate buffer (pH 6) was performed. Each antibody was incubated for 30 min and revealed using EnVision G|2 System/AP (Dako) enzyme-conjugated polymer backbone according to the manufacturer's protocol and visualized by Permanent Red Chromogen (Fas Red) included in the kit. The μm-thick sections were stained with hematoxylin and eosin (H&E). The IHC was processed using an Autostainer plus (Dako) slide processor.

Results

Flow Cytometry Analysis of 4 Specific Antibodies to NLC.

NLC were described as the Tumor Associated Macrophages (TAM) of CLL.

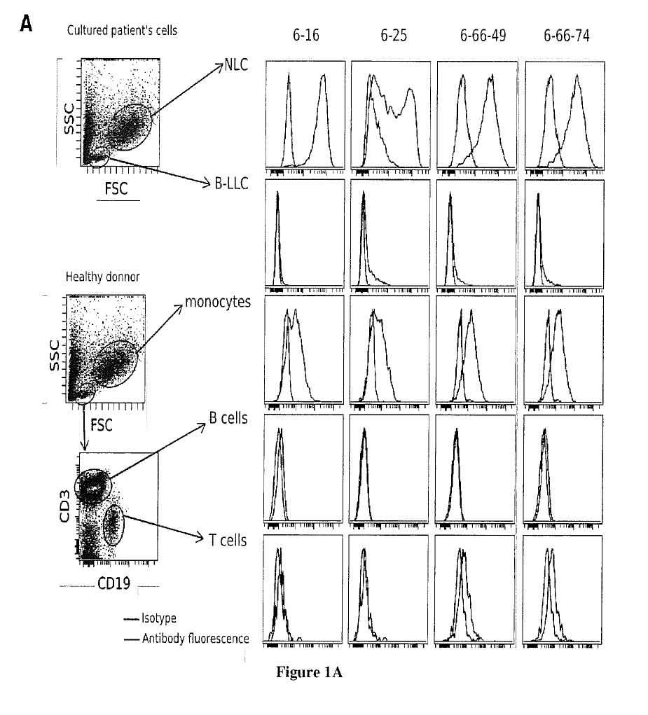

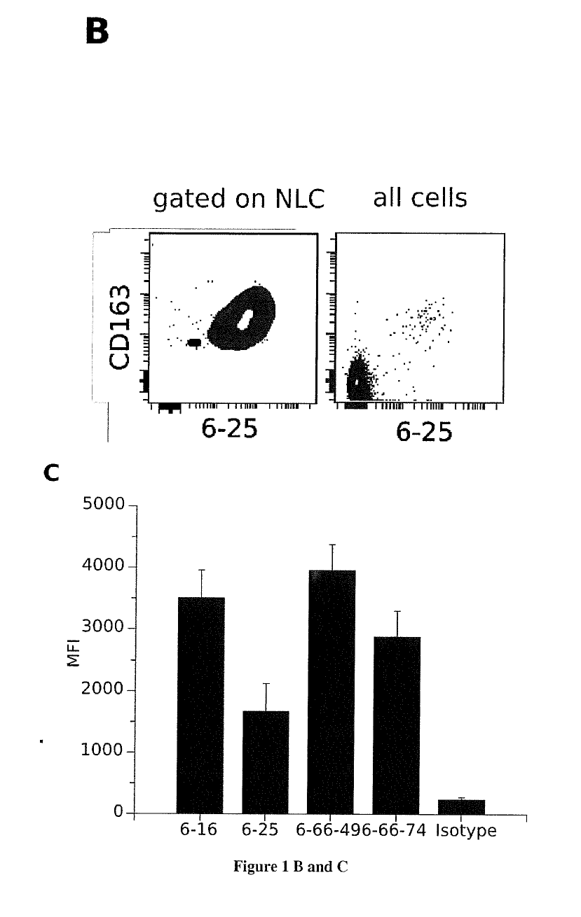

To target NLC, we decided to produce a specific antibody against NLC. Thus, we immunized mouse with purified NLC from patients with LLC. NLC were generated by the in vitro culture of Peripheral Blood Mononuclear Cells (PBMC) from patients. After 14 days, NLC were isolated then injected to mouse. About 200 hybridomas were obtained and their supernatants were tested in flow cytometry for their capability to recognize NLC from patients. Among these 200 supernatants, 4 (6-16, 6-25, 6-66-49, 6-66-74) recognized and fixed NLC, but not leukemic cells and very few monocytes from patient with LLC. FIG. 1A shows the staining with each supernatant in blue compared to the yellow isotype. These supernatants did not fixed T (CD3⁺) or B (CD19⁺) lymphocytes from healthy donors (FIG. 1 A). FIG. 1B shows the double staining of NLC with CD163, specific antibody to TAM/NLC, and 6-25 antibody, while other PBMC were not recognized by these two antibodies. These supematants contain thus specific antibodies for NLC which isotype was determined as IgG1. The mean fluorescence intensity of the 4 antibodies is represented in FIG. 1C compared to that of isoptype.

Antibodies Functionality

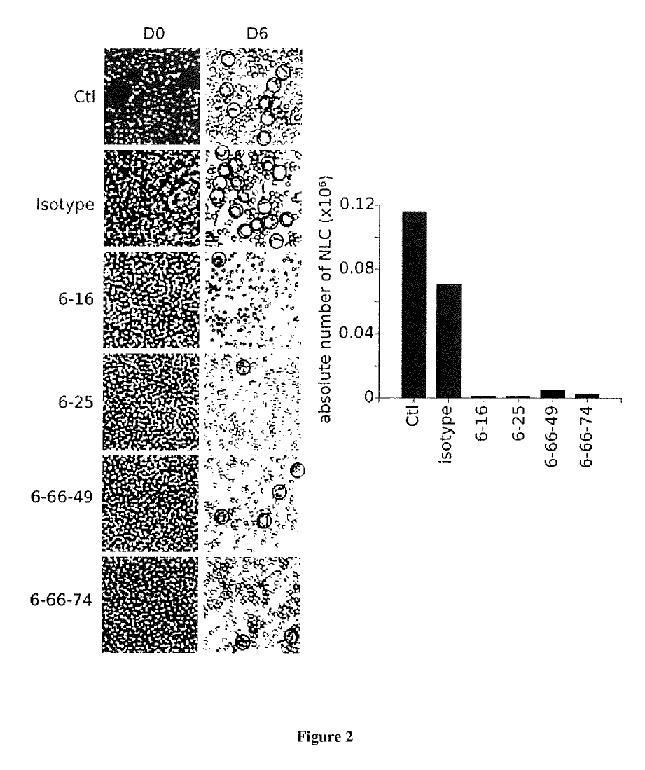

To test the antibodies functionality, we cultured PBMC from a patient with CLL with each of the 4 antibodies and showed at 6 days of culture a strong decrease of NLC number (big cells surrounded) and a decrease of the number of viable B leukemic cells compared to the culture without antibody or with isotype control (FIG. 2).

Detection of TAM in Breast Tumor

To test if the 4 antibodies were able to recognize other TAM than NLC in CLL, we performed staining in IHC of a breast tumor with the 4 antibodies. We showed that in pH6 condition, 2 of these antibodies, 6-25 and 6-66-49, produced a specific brown staining on macrophages and preferentially in the edge of the tumor, characteristic of an inflammatory area, and inside the tumor. However, a very low staining were detected away from the tumor namely in “healthy” tissue. Moreover, cancer cells or other immune cells were not stained in brown.

### EXAMPLE 3

Material & Methods

Viability of B CLL Cells and NLC after Culture with the 6-25 Antibody

PBMC isolated from blood sample of a patient with CLL were cultured for 6 days with or without 5μg/ml of purified 6-25 antibody or with 5μg/ml of isotype control (murine IgG1). Photography were obtained on a phase contrast microscope (×20) before and after depletion of B CLL. NLC were counted on the Malassez lamella. Viable leukemic cells were evaluated thanks to a staining with 7AAD and annexin V then a FACS analysis.

Western Blot

5, 10, 50 or 100 ng of recombinant sideroflexin 3 were subjected to western blotting and probed overnight at 4° C. with 10 μg/ml antibody solution with: a rabbit polyclonal anti-SFXN3 or a mouse monoclonal anti-SFXN3 or the 6-25 antibody or a mouse IgG1 isotype. After washing, membranes were incubated lh at room temperature with a solution of secondary antibody (1/10000, HRP). Revelation was then made with ECL.

Results

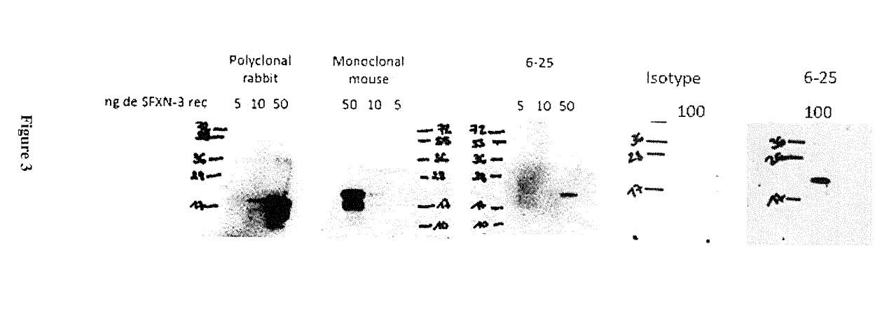

Human recombinant sideroflexin 3 was recognized in western blot by the 6-25 antibody with a band corresponding to a size of 22 kDa. Recognition of this human recombinant sideroflexin 3 was checked by western blot with two commercial antibodies, a rabbit polyclonal and a mouse monoclonal antibodies. Two bands were obtained with these two antibodies, one of which corresponded to the size of the sideroflexin 3 (22 kDa) (FIG. 3).

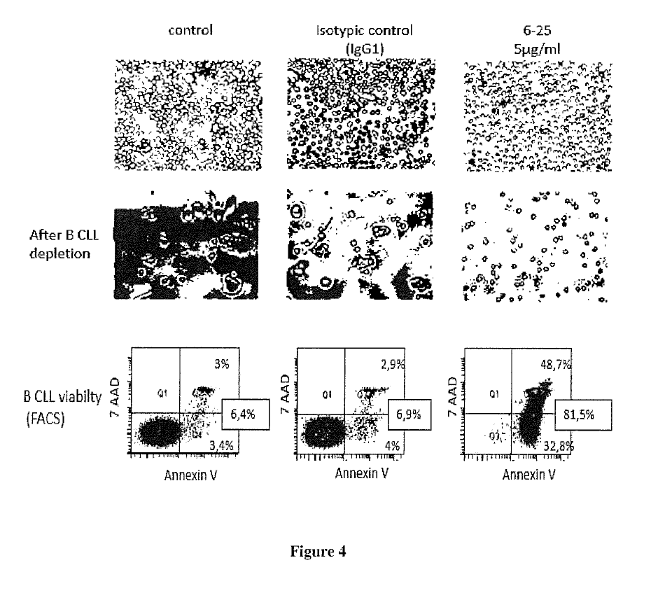

To analyze the functionality of the 6-25 antibody, we cultured PBMC of a CLL patient with or without 6-25 or isotypic control for 6 days. After 6 days, depletion of NLC was showed in the culture with 6-25. The depletion was more evident after elimination of B CCL. NLC development were not affected in the culture with the isotypic control or without antibody. Round and adherent cells was indeed visualized in these cultures compared to that with the 6-25 antibody. Then, viability of B CLL cells was analyzed in these three culture with a staining with anexin V and 7-AAD and FACS analysis. Viability of B CLL was high after 6 days of culture with or without the isotypic control, only 6.4% of dead cells. However, 81.5% of B CLL were dead after 6 days of culture in the presence of the 6-25 antibody (FIG. 4).

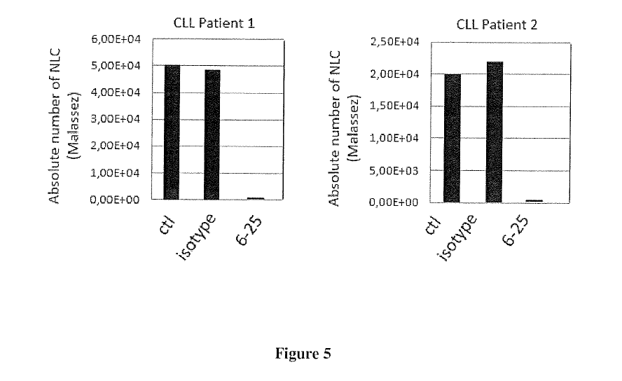

NLC were also counted in cultures of PBMC from two CLL patients with or without 6-25 or isotypic control. NLC number was close to zero in the culture with 6-25 compared to cultures with or without the isotypic control (FIG. 5).

TABLE 1
Useful nucleotide and amino acid sequences for practicing the invention

| SEQ ID | |
|---|---|
| NO | Nucleotide or amino acid sequence |
| 1 | MESKMGELPL DINIQEPRWD QSTFLGRARH FFTVTDPRNL |
| (sideroflexin | LLSGAQLEAS RNIVQNYRAG VVTPGITEDQ LWRAKYVYDS |
| 3 AA | AFHPDTGEKV VLIGRMSAQV PMNMTITGCM LTFYRKTPTV |
| sequence) | VFWQWVNQSF NAIVNYSNRS GDTPITVRQL GTAYVSATTG |
| | AVATALGLKS LTKHLPPLVG RFVPFAAVAA ANCINIPLMR |
| | QRELQVGIPV ADEAGQRLGY SVTAAKQGIF QVVISRICMA |
| | IPAMAIPPLI MDTLEKKDFL KRRPWLGAPL QVGLVGFCLV |
| | FATPLCCALF PQKSSIHISN EPELRAQIH EQNPSVEVVY YNKGL |
| 2 | ggcacctccc ttaggcgcca gggacagccg agcgttacct ggtcccgggc agcggagttc |
| (sideroflexin | tttacccacc ccagttctgg ttctgacgcc ctagctcatt ccgcaaattt agggcttggg tctggcttgt |
| 3 nucleic | tcccctccgg ctcgaaccac ctcttctctg agccgagcca gctaccgggg ctcctggaat tgccacccct |
| acid | ccctgggcac ccttgaggcc tccgtggagg gacgtcacgg ggcagagcgg gacgtgagcc |
| sequence) | tgagtttgct gcaggcgtgc tctgtgtggt ggctgggttc tgccaatccc cgtgcccacc gggtgggcgc |
| | ggccgggaag ctcctgcccc tccctgctgg tcggcgtcac gcgtgacgtc ccgcgtgatg |
| | gctgggaggg cccggcggcg acagcggagg cagagaggaa ggcggttctg agagcttcag |
| | agagcgatgg aaagcaaaat gggtgaattg cctttagaca tcaacatcca ggaacctcgc tgggaccaaa |
| | gtactttcct gggcagagcc cggcactttt tcactgttac tgatcctcga aatctgctgc tgtccggggc |
| | acagctggaa gcttctcgga acatcgtgca gaactacagg gccggcgtgg tgaccccagg |
| | gatcaccgag gaccagctgt ggagggccaa gtatgtgtat gactccgcct tccatccgga cacaggggag |
| | aaggtggtcc tgattggccg catgtcagcc caggtgccca tgaacatgac catcactggc tgcatgctca |
| | cattctacag gaagacccca accgtggtgt tctggcagtg ggtgaatcag tccttcaatg ccattgttaa |
| | ctactccaac cgcagtggtg acactcccat cactgtgagg cagctgggga cagcctatgt gagtgccacc |
| | actggagctg tggccacggc cctgggactc aaatccctca ccaagcacct gccccccttg gtcggcagat |
| | ttgtgccctt tgcagcagtg gcagctgcca actgcatcaa catccccctg atgaggcaga gagagctgca |
| | ggtgggcatc ccggtggctg atgaggcagg tcagaggctt ggctactcgg tgactgcagc |
| | caagcaggga atcttccagg tggtgatttc aagaatctgc atggcgattc ctgccatggc catcccacca |
| | ctgatcatgg acactctgga gaagaaagac ttcctgaagc gccgcccctg gctgggggca cccctgcagg |
| | tgggactggt gggcttctgc ctggtatttg caacccccct gtgctgtgcc ctattccccc agaagagctc |
| | catacacata agcaacctgg aaccagagct gagagctcag atccatgagc aaaaccccag cgttgaagtg |
| | gtctactaca acaaggggct ttgaggaggg tcagcctctg tcccctccct cacttccttg ggctgctgct |
| | ttagtggagt catgtcaccc ctaccacttg gctatctgcc tagcactggg caggggcctt ggtgggcaga |
| | tggcaattga gggtagcaac ctattagggt gggggaggga cctccataag gcttttcctc ccttctctgg |
| | tttcaaagat cagagcacat aacccctcct gtgcttgagt gtccatgcat atacatacat gatacacatg |
| | tgtatgtgta cattgggtcc tgaaagctag aagcaggcat gctagcctag tatgttctga catctggctt |
| | cccttctcag cctcatgtcc acctgcctgc cagccaggct acaggtgtga cttccttctc taaactgtta |
| | caccagccaa gttatttttg atggcacctc atcccttcta gaaataggag gagccccagg atctcaggac |
| | agagacttat agacactagt aggacaaagc gggctgaatc cttcaggttt ctgataccta gctccccaag |
| | ctgactgggc tggcagagga gaacatgttg agacaaggga ggcaggggac ttatgcatcc ctcagtgcca |
| | tcccttgtat cctggaatag ctccatttcc cctcctcctc tctaccagac aaaggagtgc ctgtgtcctg |
| | tactgccctc gctgtctccc ccaccaccct acttgacagc gtgggcatct tcaggcacag ccttgggagt |
| | tcctggtgtg ctctgacatc atgacctcaa atctaaatcc tccaatccca actccctttc ccaaacaaaa |
| | agccacagag gcagagcaag cattcccctt taagagcttc cactgcaccc cctcccaagg gacacagcgg |
| | taggaatggt gcttaaactc cacaggtatc agagagggtg taactaggac atcctcaagg gcagctaggc |
| | cccgaatgta caatgttaag acagggaatt ttgtgttcca ttgacttttt tttttttttt taatggagtt tcactatttt |
| | gcccaggctg gagtgcgatg gtgcgatctt ggctcactgc aacctctgcc tcctgggttc aagtgattct |
| | cttgcctcag tctcccgagt agtggaaatt acaggtgtgt gctaccacat cttgctagtt ttgtattttt |
| | agcagagatg ggggtttcac catgttggcc aggctagtct cgaactcctg acctcaggtg atccacctgc |
| | cttggcctcc caaagcactg ggattacaag catgagccac tgtgcccagc ctgttccact gacatttctt |
| | agacattcag caaaaccccc accttaacct cttttctttc ttgagggttg gtcctgtccc cacctccacc |
| | ctcccacccc ctggaagagg aagggcccgg gcatcagtgg ctagtccaaa taaaatatgg gcttggggat |
| | ggaatgggtg gtggtaagtt cacagagtgt agttagatcc caactcccat gacctctggc ttcagtggtg |
| | ggtggggcag ggcagatgaa agggcttcag tgggaacctc tgagagcatt ttcctgttcc ccctatcaac |
| | cgcccccagt gataacatct gtgaagccag ccattactca ataaactgca aacttgtcta aaaaaaaaaa |
| | aaaaaaaaaa aaaaaaaaaa aaaaaaaaa |

### REFERENCES

Throughout this application, various references describe the state of the art to which this invention pertains. The disclosures of these references are hereby incorporated by reference into the present disclosure.

1. A method for identifying tumor-associated macrophage (TAM) in a sample comprising the steps of i) detecting the cell surface expression of CD163, CD68, and SFXN3 markers on the cell population contained in the sample and ii) concluding that the cells expressing CD163, CD68, and SFXN3 markers are the TAMs.

2. The method according to claim 1 wherein the sample is a fluid sample selected from the group consisting of blood samples and peripheral blood mononuclear cell (PBMC) samples in suspension.

3. The method of claim 1 wherein the detection of the cell surface expression of CD163, CD68, and SFXN3 markers on the cell population contained in the fluid sample is performed with a set of antibodies specific for the markers.

4. The method of claim 1 wherein the detection of the cell surface expression of CD163, CD68, and SFXN3 markers on the cell population contained in the fluid sample is performed by flow cytometry.

5. The method according to claim 1 wherein the sample is a tissue sample selected from the group consisting of tissue sections of brain, head and neck, adrenal glands, colon, small intestines, stomach, heart, liver, skin, kidney, lung, pancreas, testis, ovary, prostate, uterus, thyroid, spleen, bladder, breast, bone marrow and lymph nodes.

6. The method of claim 5 comprising the step of staining the tissue sample with a set of antibodies specific for CD163, CD68, and SFXN3.

7. The method of claim 5 wherein the detection of the cell expression of CD163, CD68, and SFXN3 markers in the tissue sample is performed by immunochemistry or immunofluorescence.

8. A method of preventing or treating cancer in a patient, comprising
administering to the patient a therapeutically effective amount of an SFXN3 antagonist.

9. The method according to claim 8, wherein said cancer is an acute leukemia or a chronic leukemia.

10. The method according to claim 9, wherein said chronic leukemia is chronic lymphocytic leukemia.

11. The method according to claim 8, wherein said cancer is a non-Hodgkinien lymphoma or a Hodgkinien lymphoma.

12. The method according to claim 8, wherein said cancer is a solid tumor selected from the group consisting of brain, head and neck, adrenal glands, colon, small intestines, stomach, heart, liver, skin, kidney, lung, pancreas, testis, ovary, prostate, uterus, thyroid, bladder, breast, endometrial tumors, multiple myeloma, and sarcomas.

13. The method according to claim 8, wherein said antagonist is an SFXN3 neutralizing antibody or aptamer.

14. The method according to claim 8, wherein said antagonist is an inhibitor of SFXN3 gene expression.

15. A pharmaceutical composition comprising a SFXN3 antagonist and a pharmaceutically acceptable carrier.

16. The method of claim 14, wherein the inhibitor of SFXN3 gene expression is a small inhibitory RNA (siRNA), a nuclease, a ribozyme, or an antisense oligonucleotide.


SEQUENCE LISTING (2 sequences)

SEQ ID NO: 1 - protein, 324 aa, Homo sapiens
MESKMGELPLDINIQEPRWDQSTFLGRARHFFTVTDPRNLLLSGAQLEASRNIVQNYRAGVVTPGITEDQLWRAKYVYDSAFHPDTGEKVVLIGRMSAQVPMNMTITGCMLTFYRKTPTVVFWQWVNQSFNAIVNYSNRSGDTPITVRQLGTAYVSATTGAVATALGLKSLTKHLPPLVGRFVPFAAVAAANCINIPLMRQRELQVGIPVADEAGQRLGYSVTAAKQGIFQVVISRICMAIPAMAIPPLIMDTLEKKDFLKRRPWLGAPLQVGLVGFCLVFATPLCCALFPQKSSIHISNEPELRAQIHEQNPSVEVVYYNKGL

SEQ ID NO: 2 - DNA, 3129 bp, Homo sapiens
ggcacctcccttaggcgccagggacagccgagcgttacctggtcccgggcagcggagttctttacccaccccagttctggttctgacgccctagctcattccgcaaatttagggcttgggtctggcttgttcccctccggctcgaaccacctcttctctgagccgagccagctaccggggctcctggaattgccacccctccctgggcacccttgaggcctccgtggagggacgtcacggggcagagcgggacgtgagcctgagtttgctgcaggcgtgctctgtgtggtggctgggttctgccaatccccgtgcccaccgggtgggcgcggccgggaagctcctgcccctccctgctggtcggcgtcacgcgtgacgtcccgcgtgatggctgggagggcccggcggcgacagcggaggcagagaggaaggcggttctgagagcttcagagagcgatggaaagcaaaatgggtgaattgcctttagacatcaacatccaggaacctcgctgggaccaaagtactttcctgggcagagcccggcactttttcactgttactgatcctcgaaatctgctgctgtccggggcacagctggaagcttctcggaacatcgtgcagaactacagggccggcgtggtgaccccagggatcaccgaggaccagctgtggagggccaagtatgtgtatgactccgccttccatccggacacaggggagaaggtggtcctgattggccgcatgtcagcccaggtgcccatgaacatgaccatcactggctgcatgctcacattctacaggaagaccccaaccgtggtgttctggcagtgggtgaatcagtccttcaatgccattgttaactactccaaccgcagtggtgacactcccatcactgtgaggcagctggggacagcctatgtgagtgccaccactggagctgtggccacggccctgggactcaaatccctcaccaagcacctgccccccttggtcggcagatttgtgccctttgcagcagtggcagctgccaactgcatcaacatccccctgatgaggcagagagagctgcaggtgggcatcccggtggctgatgaggcaggtcagaggcttggctactcggtgactgcagccaagcagggaatcttccaggtggtgatttcaagaatctgcatggcgattcctgccatggccatcccaccactgatcatggacactctggagaagaaagacttcctgaagcgccgcccctggctgggggcacccctgcaggtgggactggtgggcttctgcctggtatttgcaacccccctgtgctgtgccctattcccccagaagagctccatacacataagcaacctggaaccagagctgagagctcagatccatgagcaaaaccccagcgttgaagtggtctactacaacaaggggctttgaggagggtcagcctctgtcccctccctcacttccttgggctgctgctttagtggagtcatgtcacccctaccacttggctatctgcctagcactgggcaggggccttggtgggcagatggcaattgagggtagcaacctattagggtgggggagggacctccataaggcttttcctcccttctctggtttcaaagatcagagcacataacccctcctgtgcttgagtgtccatgcatatacatacatgatacacatgtgtatgtgtacattgggtcctgaaagctagaagcaggcatgctagcctagtatgttctgacatctggcttcccttctcagcctcatgtccacctgcctgccagccaggctacaggtgtgacttccttctctaaactgttacaccagccaagttatttttgatggcacctcatcccttctagaaataggaggagccccaggatctcaggacagagacttatagacactagtaggacaaagcgggctgaatccttcaggtttctgatacctagctccccaagctgactgggctggcagaggagaacatgttgagacaagggaggcaggggacttatgcatccctcagtgccatcccttgtatcctggaatagctccatttcccctcctcctctctaccagacaaaggagtgcctgtgtcctgtactgccctcgctgtctcccccaccaccctacttgacagcgtgggcatcttcaggcacagccttgggagttcctggtgtgctctgacatcatgacctcaaatctaaatcctccaatcccaactccctttcccaaacaaaaagccacagaggcagagcaagcattcccctttaagagcttccactgcaccccctcccaagggacacagcggtaggaatggtgcttaaactccacaggtatcagagagggtgtaactaggacatcctcaagggcagctaggccccgaatgtacaatgttaagacagggaattttgtgttccattgacttttttttttttttttaatggagtttcactattttgcccaggctggagtgcgatggtgcgatcttggctcactgcaacctctgcctcctgggttcaagtgattctcttgcctcagtctcccgagtagtggaaattacaggtgtgtgctaccacatcttgctagttttgtatttttagcagagatgggggtttcaccatgttggccaggctagtctcgaactcctgacctcaggtgatccacctgccttggcctcccaaagcactgggattacaagcatgagccactgtgcccagcctgttccactgacatttcttagacattcagcaaaacccccaccttaacctcttttctttcttgagggttggtcctgtccccacctccaccctcccaccccctggaagaggaagggcccgggcatcagtggctagtccaaataaaatatgggcttggggatggaatgggtggtggtaagttcacagagtgtagttagatcccaactcccatgacctctggcttcagtggtgggtggggcagggcagatgaaagggcttcagtgggaacctctgagagcattttcctgttccccctatcaaccgcccccagtgataacatctgtgaagccagccattactcaataaactgcaaacttgtctaaaaaaaaaaaaaaaaaaaaaaaaaaaaaaaaaaaaaaaa